SEQUENCE LISTING (36 sequences)

SEQ ID NO: 1 - DNA, 20 bp, Artificial
  Synthetic PCR primer for GAPDH - forward sequence
tgcccagaacatcatccctg

SEQ ID NO: 2 - DNA, 20 bp, Artificial
  Synthetic PCR primer for GAPDH - reverse sequence
tcagatccacgacggacaca

SEQ ID NO: 3 - DNA, 20 bp, Artificial
  Synthetic PCR primer for ARP2 - forward sequence
cacatcttcccagctttggt

SEQ ID NO: 4 - DNA, 20 bp, Artificial
  Synthetic PCR primer for ARP2 - reverse sequence
cagctcacttgcctcatcac

SEQ ID NO: 5 - DNA, 20 bp, Artificial
  Synthetic PCR primer for ARP3 - forward sequence
caggctgttcttgccttagc

SEQ ID NO: 6 - DNA, 20 bp, Artificial
  Synthetic PCR primer for ARP3 - reverse sequence
atccttcagccacaggaatg

SEQ ID NO: 7 - DNA, 20 bp, Artificial
  Synthetic PCR primer for ARPC1a- forward sequence
gaggattaaccgtgcagcca

SEQ ID NO: 8 - DNA, 20 bp, Artificial
  Synthetic PCR primer for ARPC1a- reverse sequence
cttgctcacccaccagtcat

SEQ ID NO: 9 - DNA, 20 bp, Artificial
  Synthetic PCR primer for ARPC2- forward sequence
caccgcccttgtttctcctt

SEQ ID NO: 10 - DNA, 20 bp, Artificial
  Synthetic PCR primer for ARPC2- reverse sequence
gcttctggtttgtttccggc

SEQ ID NO: 11 - DNA, 20 bp, Artificial
  Synthetic PCR primer for ARPC3- forward sequence
ccatttatgccaagcctgcg

SEQ ID NO: 12 - DNA, 20 bp, Artificial
  Synthetic PCR primer for ARPC3- reverse sequence
gtccaccacttgctgggttt

SEQ ID NO: 13 - DNA, 20 bp, Artificial
  Synthetic PCR primer for ARPC4- forward sequence
ctctccgcccctaccttagt

SEQ ID NO: 14 - DNA, 20 bp, Artificial
  Synthetic PCR primer for ARPC4- reverse sequence
tgctactcctgacttcgacc

SEQ ID NO: 15 - DNA, 20 bp, Artificial
  Synthetic PCR primer for ARPC5- forward sequence
gaaggtggacgtggacgaat

SEQ ID NO: 16 - DNA, 20 bp, Artificial
  Synthetic PCR primer for ARPC5- reverse sequence
gctgtcatgtttccttgccg

SEQ ID NO: 17 - DNA, 21 bp, Artificial
  Synthetic PCR primer for Colla1- forward sequence
ccagtggcggttatgacttca

SEQ ID NO: 18 - DNA, 21 bp, Artificial
  Synthetic PCR primer for Colla1- reverse sequence
gctgcggatgttctcaatctg

SEQ ID NO: 19 - DNA, 20 bp, Artificial
  Synthetic PCR primer for Lox - forward sequence
agggcggatgtcagagacta

SEQ ID NO: 20 - DNA, 20 bp, Artificial
  Synthetic PCR primer for Lox - reverse sequence
aatccctgtgtgtgtgcagt

SEQ ID NO: 21 - DNA, 17 bp, Artificial
  Synthetic PCR primer for Loxl2 - forward sequence
cctccctcccgctttca

SEQ ID NO: 22 - DNA, 21 bp, Artificial
  Synthetic PCR primer for Loxl2 - reverse sequence
caagtgtgcagtcctgggttt

SEQ ID NO: 23 - DNA, 20 bp, Artificial
  Synthetic PCR primer for Tg2 - forward sequence
ggaggagcgacgggaatatg

SEQ ID NO: 24 - DNA, 20 bp, Artificial
  Synthetic PCR primer for Tg2 - reverse sequence
attccatcctcgaactgccc

SEQ ID NO: 25 - DNA, 20 bp, Artificial Sequence
  Synthetic PCR primer for Col1a2 - forward sequence
ctgatggcagagctggtgta

SEQ ID NO: 26 - DNA, 20 bp, Artificial Sequence
  Synthetic PCR primer for Col1a2 - reverse sequence
atgttgccagcttcacctct

SEQ ID NO: 27 - DNA, 18 bp, Artificial Sequence
  Synthetic PCR primer for UBC - forward sequence
cagttggtcctgcgcttg

SEQ ID NO: 28 - DNA, 23 bp, Artificial Sequence
  Synthetic PCR primer for UBC - reverse sequence
ttttttgggaatgcaacaacttt

SEQ ID NO: 29 - DNA, 20 bp, Artificial Sequence
  Synthetic PCR primer for ARPC2 - forward sequence
tttcatcccgctaatcttgg

SEQ ID NO: 30 - DNA, 20 bp, Artificial Sequence
  Synthetic PCR primer for ARPC2 - reverse sequence
gggagaaaagggaagtgagg

SEQ ID NO: 31 - DNA, 18 bp, Artificial Sequence
  Synthetic PCR primer for ACTA2 - forward sequence
gaagagcatcccaccctg

SEQ ID NO: 32 - DNA, 18 bp, Artificial Sequence
  Synthetic PCR primer for ACTA2 - reverse sequence
attttctcccggttggcc

SEQ ID NO: 33 - DNA, 22 bp, Artificial Sequence
  Synthetic PCR primer for COL1A1 - forward sequence
ttgagactcagccacccagagt

SEQ ID NO: 34 - DNA, 21 bp, Artificial Sequence
  Synthetic PCR primer for COL1A1 - reverse sequence
cagttcttggctgggtgtttt

SEQ ID NO: 35 - DNA, 20 bp, Artificial Sequence
  Synthetic PCR primer for COL1A2 - forward sequence
agcaggtccttggaaacctt

SEQ ID NO: 36 - DNA, 20 bp, Artificial Sequence
  Synthetic PCR primer for COL1A2 - reverse sequence
gaaaaggagttggacttggc

(19) United States
(12) Patent Application Publication
(10) Pub. No.: US 2020/0237864 A1
(43) Pub. Date: Jul. 30, 2020

(54) METHODS AND COMPOSITIONS FOR TREATING FIBROTIC INTERSTITIAL LUNG DISEASE

(71) Applicants: INSERM (INSTITUT NATIONAL DE LA SANTÉ ET DE LA RECHERCHE MÉDICALE), Paris (FR); CENTRE HOSPITALIER RÉGIONAL UNIVERSITAIRE DE TOURS, Tours (FR); UNIVERSITÉ DE TOURS FRANÇOIS RABELAIS, Tours (FR)

(72) Inventor: Laurent PLANTIER, Tours-Cedex 1 (FR)

(21) Appl. No.: 16/754,826

(22) PCT Filed: Oct. 9, 2018
(86) PCT No.: PCT/EP2018/077451

§ 371 Date: Apr. 9, 2020

(30) Foreign Application Priority Data
Oct. 10, 2017 (EP) 17306360.3

(51) Int. Cl. A61K 38/17; A61K 31/4025; A61K 31/426; A61K 31/404; A61P 11/00

(52) U.S. Cl. CPC A61K 38/1719; A61K 31/4025; A61P 11/00; A61K 31/404; A61K 31/426

(57) ABSTRACT

In the present invention, inventors study in fibrotic ILD/IPF mechanism, the role of Arp2/3 multiprotein complex which regulates the branched polymerization of the actin cytoskeleton, and may play key roles in mechanotranduction. Inventors demonstrated that the expression of the Arp2/3 complex is increased in IPF lung mesenchymal cells and in mouse lungs during fibrogenesis, and that the inhibition of Arp2/3 complex with the small molecule CK666 blocks collagen expression in the lungs and prevents the development of bleomycin-induced lung fibrosis in mice. Accordingly the present invention relates to an inhibitor of Arp2/3 complex activity or expression for use in a method for treating Fibrotic interstitial lung diseases (ILD) such as idiopathic pulmonary fibrosis in a subject in need thereof.

### FIELD OF THE INVENTION

The present invention relates methods and pharmaceutical compositions for treating fibrotic interstitial lung diseases such as idiopathic pulmonary fibrosis.

### BACKGROUND OF THE INVENTION

Fibrotic interstitial lung diseases (ILD) are a group of chronic lung diseases where progressive fibrosis of alveolar regions leads to respiratory insufficiency. Fibrotic ILD carry a poor prognosis and therapeutic options are lacking for the satisfactory treatment of fibrotic ILD (“ERS White Book,” n.d.). Patients with fibrotic ILD related to inflammatory of auto-immune disease benefit from anti-inflammatory of immunosuppressive therapy (Adegunsoye and Strek 2016), while these drugs are detrimental in patients with Idiopathic pulmonary fibrosis (IPF), the most severe form of fibrotic ILD (Idiopathic Pulmonary Fibrosis Clinical Research Network et al. 2012). Although two antifibrotic drugs, pirfenidone and nintedanib, slow the decline in lung function and reduce the risk of death in IPF (Noble et al. 2011; Richeldi et al. 2014; Canestaro et al. 2016), neither halts the progress of disease. Thus, there is a need for novel treatments of fibrotic ILD.

Fibrosis is defined by the accumulation of an abnormally stiff extracellular matrix rich in fibrillary collagens, which destroys the normal architecture of the lung and profoundly alters its function. A key pathogenic determinant of fibrotic ILD is the differentiation of resident lung mesenchymal cells such as pericytes and alveolar fibroblasts into collagen-secreting lung myofibroblasts (Crestani et al. 2013). The mechanisms driving the profibrotic differentiation of lung mesenchymal cells have been best studied in IPF. These include cell-autonomous determinants such as epigenetic alterations of the chromatin (S. K. Huang et al. 2010) and expression of profibrotic non-coding RNAs (Pottier et al. 2014), epithelial-mesenchymal interactions triggered by alveolar epithelial lesions (Selman and Pardo 2006) and mediated in part by the release of soluble pro-fibrotic mediators such as the archetypal TGF-Beta (Khalil et al. 1991), and matrix-mesenchyme interactions. Matrix-mesenchyme interactions in the setting of fibrogenesis comprise indirect signals through membrane-bound mediators (Lepparanta et al. 2012) or matrix degradation peptides with intrinsic signaling activity (Shiratsuchi et al. 2010), and direct mechanotransduction where mechanical cues transmitted by the actin cytoskeleton drive changes in cell phenotype (Duscher et al. 2014).

Recent studies showed the potential key role of actin cytoskeleton dynamics in fibrogenic processes in the lung. The actin cytoskeleton connects matrix-bound focal adhesions with the nucleoskeleton (Simon and Wilson 2011) and plays key roles in mechanotransduction. Under control by small GTPases such as Rho-associated coiled-coil forming protein kinase (ROCK) 1 and 2, the actin cytoskeleton organizes as a contractile structure (Pellegrin and Mellor 2007) and induces the cytoplasmic-to-nuclear shuttling or activation of intracellular proteins such as the fibroblast growth factor receptor (Sandilands et al. 2007) or pro-fibrotic transcription factors such as YAP/TAZ (Dupont et al. 2011), MRTF-A (Zhao et al. 2007), SRF (Johnson et al. 2014) and MKL1 (Zhou et al. 2013). Mechanotransduction is required for the TGF-B-induced profibrotic differentiation of lung fibroblasts in vitro (Zhou et al. 2013; Marinković, Liu, and Tschumperlin 2013) while pharmacological inhibition of ROCK prevents the development of bleomycin-induced lung fibrosis in mice (Zhou et al. 2013). In further support of the hypothesis that actin-related mechanotransduction pathways are critical for the profibrotic differentiation of lung mesenchymal cells, the actin related protein 2/3 (Arp2/3) complex subunit 2 (ARPC2) protein is required for collagen-1 expression by lung fibroblasts in vitro (Melboucy-Belkhir et al. 2014). The Arp2/3 complex, which comprises 7 distinct proteins, is a key regulator of the actin cytoskeleton. The Arp2/3 complex catalyzes actin nucleation and allows the branched polymerization of actin monomers. The Arp2/3 complex may be a key player in mechanotransduction (Wu et al. 2012, 3).

### SUMMARY OF THE INVENTION

In the present invention, inventors study in fibrotic ILD/IPF mechanism the role of Arp2/3 multiprotein complex which regulates the branched polymerization of the actin cytoskeleton, and may play key roles in mechanotranduction. Inventors demonstrated that the expression of the Arp2/3 complex is increased in IPF lung mesenchymal cells and in mouse lungs during fibrogenesis, and that the inhibition of Arp2/3 complex with the small molecule CK666 (Hetrick et al. 2013) blocks collagen expression in the lungs and prevents the development of bleomycin-induced lung fibrosis in mice.

Accordingly the present invention relates to an inhibitor of Arp2/3 complex activity or expression for use in a method for treating Fibrotic interstitial lung diseases (ILD) in a subject in need thereof.

The present invention also relates to a method for screening a plurality of candidate compounds useful for treating Fibrotic interstitial lung diseases (ILD) comprising the steps consisting of (a) testing each of the candidate compounds for its ability to inhibit Arp 2/3 complex activity or expression and (b) and positively selecting the candidate compounds capable of inhibiting said Arp 2/3 complex activity or expression.

### DETAILED DESCRIPTION OF THE INVENTION

In the present study the inventors demonstrate 1) that the Arp2/3 complex is overexpressed in lung fibroblasts cultured from patients with idiopathic pulmonary fibrosis (IPF) and in the lungs of mice with bleomycin-induced pulmonary fibrosis, 2) that inhibition of the Arp2/3 complex with the small molecule CK666 prevents the development of bleomycin-induced pulmonary fibrosis in C57BL/6 mice. In mice, CK666 (delivered intraperitoneally) reduced lung levels of soluble procollagen-1 and insoluble collagen despite preserved levels of collagen-1 mRNA, suggesting that the Arp2/3 complex is required for the translation of collagen-1 mRNA to protein. Altogether, these results indicate that inhibition of the Arp2/3 complex inhibits lung fibrosis in vivo and represents an attractive new therapy for IPF and other fibrotic interstitial lung diseases.

Accordingly, the present invention relates to an inhibitor of Arp 2/3 complex activity or expression for use in a method for treating Fibrotic interstitial lung diseases (ILD) such as idiopathic pulmonary fibrosis (IPF) in a subject in need thereof.

As used herein, the term “Fibrotic interstitial lung diseases” or “Fibrotic ILD” or “Pulmonary Fibrosis” refers to a group of diseases affecting the pulmonary interstitium (the tissue and space between the air sacs of the lungs and lung capillaries). Fibrotic ILD refers to more than 200 chronic lung disorders. With fibrotic ILD, the tissue between the air sacs of the lungs (the interstitium) and the lung capillaries is affected by the accumulation of extracellular matrix rich in fibrillary collagens (fibrosis). It concerns alveolar epithelium, pulmonary capillary endothelium, basement membrane, perivascular and perilymphatic tissues.

In a specific embodiment ILD is selected from the list consisting of Idiopathic pulmonary fibrosis (IPF), idiopathic non-specific interstitial pneumonia (NSIP), fibrotic ILD associated with inflammatory rheumatic disease such as rheumatoid arthritis, fibrotic ILD associated with autoimmune connective tissue disease such as systemic sclerosis, Sjögren's syndrome, undifferentiated connective tissue disease, chronic hypersensitivity pneumonitis and lung fibrosis associated with unresolving acute respiratory distress syndrome (ARDS).

In a preferred embodiment ILD is Idiopathic pulmonary fibrosis (IPF).

As used herein, the terms “Idiopathic pulmonary fibrosis” or “IPF” means an interstitial lung disease for which no obvious cause can be identified (idiopathic), and which is associated with typical findings both radiographic (basal and pleural based fibrosis with honeycombing) and pathologic (usual interstitial pneumonia pattern comprising temporally and spatially heterogeneous fibrosis, histopathologic honeycombing and fibroblastic foci).

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a subject, and includes: (a) increasing survival time; (b) decreasing the risk of death due to the disease; (c) preventing the disease from occurring in a subject which may be predisposed to the disease but has not yet been diagnosed as having it; (d) inhibiting the disease, i.e., arresting its development (e.g., reducing the rate of disease progression); and (e) relieving the disease, i.e., causing regression of the disease.

The terms “subject,” and “patient,” used interchangeably herein, refer to a mammal, particularly a human who has been previously diagnosed with Fibrotic interstitial lung diseases such as idiopathic pulmonary fibrosis or who is at risk for having or developing idiopathic pulmonary fibrosis. Typically, a diagnosis of idiopathic pulmonary fibrosis may be made after lung biopsy or by using high resolution computed tomography (HRCT).

As used herein the term ‘or “Arp2/3 complex” has its general meaning in the art. Arp 2/3 complex comprises 7 distinct proteins, and is a key regulator of the actin cytoskeleton. The Arp2/3 complex catalyzes actin nucleation and allows the branched polymerization of actin monomers. The Arp2/3 complex may be a key player in mechanotransduction (Wu et al. 2012, 3). The Arp2/3 complex is composed of seven subunits: Arp2, Arp3, p41/ARPC1, p34/ARPC2, p21/ARPC3, p20/ARPC4, p16/ARPCS. The subunits Arp2 and Arp3 closely resemble monomeric actin allowing for a thermodynamically stable actin-like dimer. p41/ARPC1 has been proposed to interact with nucleation promoting factors (NPFs) because it is only known to have minor contacts with the mother filament and there is a major loss of nucleation efficiency in the absence of p41/ARPC1. p34/ARPC2 and p20/ARPC4 dimerize to form a structural backbone that mediates the interaction with the mother filament. p21/ARPC3 forms a bridge between Arp3 and the mother filament (Robinson et al. 2001) increasing nucleation efficiency. p16/ARPCS tethers Arp2 to the rest of the complex.

Accordingly the Arp2/3 complex is composed of Arp2, Arp3, p41/ARPC1, p34/ARPC2, p21/ARPC3, p20/ARPC4, p16/ARPC5 subunits.

inhibitor of Arp2/3 complex activity

An “inhibitor of Arp2/3 complex activity” has its general meaning in the art, and refers to a compound (natural or not) which has the capability of reducing or suppressing the biological activity of Arp2/3 complex or of one of its subunit. In the present application, said compound inhibits or reduces profibrotic differentiation of lung cells induced by the actin polymerisation initiated by the Arp 2/3 complex. For example the compound may block the interaction of Arp2/3 complex with actin, or may bind to Arp2/3 complex in manner that Arp2/3 complex is not able to bind to actin (mother filament or monomer), may inhibit the formation of Arp 2/3 complex, or may inhibit the expression of either Arp2, Arp3, p41/ARPC1, p34/ARPC2, p21/ARPC3, p20/ARPC4, p16/ARPC5 subunits. Typically, said inhibitor is a small organic molecule or a biological molecule (e.g. peptides, lipid, aptamer, antibody . . . ).

By “biological activity” of of Arp2/3 complex is meant inducing profibrotic differentiation of lung cells associated with the actin polymerisation.

Tests for determining the capacity of a compound to be inhibitor of Arp2/3 complex activity are well known to the person skilled in the art. In a preferred embodiment, the inhibitor specifically binds to Arp2/3 complex in a sufficient manner to inhibit the biological activity of Arp2/3 complex. Binding to Arp2/3 complex and inhibition of the biological activity of Arp2/3 complex may be determined by any competing assays well known in the art. For example the assay may consist in determining the ability of the agent to be tested as Arp2/3 complex activity inhibitor to bind to Arp2/3 complex. The binding ability is reflected by the Kd measurement. The term “KD”, as used herein, is intended to refer to the dissociation constant, which is obtained from the ratio of Kd to Ka (i.e. Kd/Ka) and is expressed as a molar concentration (M). KD values for binding biomolecules can be determined using methods well established in the art. In specific embodiments, an inhibitor that “specifically binds to Arp2/3 complex ” is intended to refer to an inhibitor that binds to human Arp2/3 complex polypeptide with a KD of 1 μM or less, 100 nM or less, 10 nM or less, or 3 nM or less. Then a competitive assay may be settled to determine the ability of the agent to inhibit biological activity of Arp2/3 complex. The functional assays may be envisaged such evaluating the ability to induce or inhibit the profibrotic differentiation of lung mesenchymal cells (see example with CK666 compound and FIGS. 4).

The skilled in the art can easily determine whether an Arp2/3 complex activity inhibitor neutralizes, blocks, inhibits, abrogates, reduces or interferes with a biological activity of the Arp2/3 complex. To check whether the Arp2/3 complex activity inhibitor binds to Arp2/3 complex and/or inhibits lung fibrosis in the same way than the initially characterized CK666 compound may be performed with each inhibitor. For instance lung fibrosis in vivo can be measured by the Sircol Insoluble collagen Assay (level of expression of Collagen 1 is the marker of profibrotic differentiation of mesenchymal cells) on lung tissue as described in the Examples section (S.-Y. Park et al. 2015)(J. Huang et al. 2016).

In a particular embodiment, the activity inhibitor according to the invention is a small organic molecule such as CK-636 (Cas No. 442632-72-6) or CK-548 (see Nolen B. J. et al “Characterization of two classes of small molecule inhibitors of Arp2/3 complex” Nature. 2009 Aug. 20; 460(7258): 1031-1034.) or the derived compounds CK666 (CAS Number 442633-00-3) or CK-869 (CAS 170930-46-8) (Hetrick et al. 2013) or synthetics triterpenoids such as 2-cyano-3,12-dioxooleana-1,9-dien-28-oic acid (CDDO)-Im and CDDO-Me (To, Shilton, and Di Guglielmo 2010).

In a particular embodiment, the activity inhibitor according to the invention is a protein or peptide such as Arpin or derived peptide (see WO2015033267 and Dang et al. 2013).

In a particular embodiment, the activity inhibitor according to the invention is an antibody or portions thereof.

In this embodiment, the activity inhibitor of Arp2/3 complex is an antibody (the term including antibody fragment or portion) that can bind to Arp2/3 complex in the cells and block its biological activity.

In preferred embodiment, the activity inhibitor of Arp2/3 complex may consist in an antibody directed against the Arp2/3 complex (or one of its subunit), in such a way that said antibody impairs Arp2/3 complex activity (“neutralizing antibody”).

Then, for this invention, neutralizing antibody of Arp2/3 complex are selected as above described for their capacity to (i) bind to Arp2/3 complex and/or (ii) inhibiting the profibrotic differentiation of lung cells associated with the actin polymerisation by the Arp2/3 complex.

In one embodiment of the antibodies or portions thereof described herein, the antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion of the antibody comprises a F(ab′)2 portion of the antibody. In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

Antibodies are prepared according to conventional methodology. Monoclonal antibodies may be generated using the method of Kohler and Milstein (Nature, (1975) 256:495, 1975). To prepare monoclonal antibodies useful in the invention, a mouse or other appropriate host animal is immunized at suitable intervals (e.g., twice-weekly, weekly, twice-monthly or monthly) with antigenic forms of sideroflexin 3. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the recombinant Arp2/3 complex (or one of its subunit) may be provided by expression with recombinant cell lines. Recombinant form of Vasohibin and/or SVBP may be provided using any previously described method. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

As the Arp2/3 complex is an intracellular target, the antibody of the invention acting as an activity inhibitor could be an antibody fragment without Fc fragment.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4.

In an embodiment, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “nanobody®”. According to the invention, sdAb can particularly be llama sdAb.

Inhibitor of Arp2/3 complex expression

An “inhibitor of Arp2/3 complex expression” refers to a natural or synthetic compound that has a biological effect to inhibit or significantly reduce the expression of at least one gene encoding for at least one different subunit of Arp 2/3 Complex.

In a particular embodiment inhibitor of Arp2/3 complex expression is selected from the group consisting of inhibitor of expression of Arp2, Arp3, p41/ARPC1, p34/ARPC2, p21/ARPC3, p20/ARPC4 or p16/ARPC5 subunit.

TABLE 1
Arp2/3 complex subunit

| | Gene | |
|---|---|---|
| Arp2/3 | ID | |
| complex | (human | |
| subunit | gene) | NCBI accession Number |
| Arp2 | 10097 | Isoform a: NM_001005386/NP_001005386 |
| | | Isoform b: NM_005722/NP_005713 |
| Arp3 | 10096 | Isoform 1: NM_005721/NP_005712 |
| | | Isoform 2: NM_001277140/NP_001264069 |
| p41/ARPC1(B) | 10095 | NM_005720.3/NP_005711.1 |
| p34/ARPC2 | 10109 | Variant 1: NM_152862/NP_690601 |
| | | Variant 2: NM_005731/NP_005722 |
| p21/ARPC3 | 10094 | Isoform 1: NM_001278556/NP_001265485 |
| | | Isoform 2: NM_001287222/NP_001274151 |
| p20/ARPC4 | 10093 | Isoform a: NM_005718/NP_005709 |
| | | Isoform b1: NM_001024959/NP_001020130 |
| | | Isoform b2: NM_001024960/NP_001020131 |
| | | Isoform c: NM_001198780/NP_001185709 |
| p16/ARPC5 | 10092 | Isoform 1: NM_005717/NP_005708 |
| | | Isoform 2: NM_001270439/NP_001257368 |

As shown in the Example section, the Arp2/3 complex subunits overexpressed at mRNA level in IPF lung fibroblasts compared to the controls lung fibroblasts are ARPC2, ARPC3 and ARPC5.

Inhibitors of expression for use in the present invention may be based on anti-sense oligonucleotide constructs. Anti-sense oligonucleotides, including anti-sense RNA molecules and anti-sense DNA molecules, would act to directly block the translation of Arp 2/3 complex mRNAs by binding thereto and thus preventing protein translation or increasing mRNA degradation, thus decreasing the level of Arp 2/3 complex, and thus activity, in a cell. For example, antisense oligonucleotides of at least about 15 bases and complementary to unique regions of the mRNA transcript sequence encoding Arp 2/3 complex can be synthesized, e.g., by conventional phosphodiester techniques and administered by e.g., intravenous injection or infusion, intratracheal instillation, or in the form of inhaled aerosols. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of expression for use in the present invention. Arp 2/3 complex gene expression can be reduced by contacting a subject or cell with a small double stranded RNA (dsRNA), or a vector or construct causing the production of a small double stranded RNA, such that Arp 2/3 complex gene expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 1/36646, WO 99/32619, and WO 1/68836). All or part of the phosphodiester bonds of the siRNAs of the invention are advantageously protected. This protection is generally implemented via the chemical route using methods that are known by art. The phosphodiester bonds can be protected, for example, by a thiol or amine functional group or by a phenyl group. The 5′- and/or 3′-ends of the siRNAs of the invention are also advantageously protected, for example, using the technique described above for protecting the phosphodiester bonds. The siRNAs sequences advantageously comprises at least twelve contiguous dinucleotides or their derivatives.

As used herein, the term “siRNA derivatives” with respect to the present nucleic acid sequences refers to a nucleic acid having a percentage of identity of at least 90% with Arp2/3 complex subunit or fragment thereof, preferably of at least 95%, as an example of at least 98%, and more preferably of at least 98%.

As used herein, “percentage of identity” between two nucleic acid sequences, means the percentage of identical nucleic acid, between the two sequences to be compared, obtained with the best alignment of said sequences, this percentage being purely statistical and the differences between these two sequences being randomly spread over the nucleic acid acids sequences. As used herein, “best alignment” or “optimal alignment”, means the alignment for which the determined percentage of identity (see below) is the highest. Sequences comparison between two nucleic acids sequences are usually realized by comparing these sequences that have been previously align according to the best alignment; this comparison is realized on segments of comparison in order to identify and compared the local regions of similarity. The best sequences alignment to perform comparison can be realized, beside by a manual way, by using the global homology algorithm developed by SMITH and WATERMAN (Ad. App. Math., vol.2, p:482, 1981), by using the local homology algorithm developped by NEDDLEMAN and WUNSCH (J. Mol. Biol., vol.48, p:443, 1970), by using the method of similarities developed by PEARSON and LIPMAN (Proc. Natl. Acd. Sci. USA, vol.85, p:2444, 1988), by using computer software using such algorithms (GAP, BESTFIT, BLAST P, BLAST N, FASTA, TFASTA in the Wisconsin Genetics software Package, Genetics Computer Group, 575 Science Dr., Madison, Wis. USA), by using the MUSCLE multiple alignment algorithms (Edgar, Robert C., Nucleic Acids Research, vol. 32, p:1792, 2004). To get the best local alignment, one can preferably used BLAST software. The identity percentage between two sequences of nucleic acids is determined by comparing these two sequences optimally aligned, the nucleic acids sequences being able to comprise additions or deletions in respect to the reference sequence in order to get the optimal alignment between these two sequences. The percentage of identity is calculated by determining the number of identical position between these two sequences, and dividing this number by the total number of compared positions, and by multiplying the result obtained by 100 to get the percentage of identity between these two sequences.

shRNAs (short hairpin RNA) can also function as inhibitors of expression for use in the present invention.

Ribozymes can also function as inhibitors of expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of ARP 2/3 COMPLEX mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable.

Both antisense oligonucleotides and ribozymes useful as inhibitors of expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and preferably cells expressing ARP 2/3 COMPLEX. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991).

Preferred viruses for certain applications are the adenoviruses and adeno-associated (AAV) viruses, which are double-stranded DNA viruses that have already been approved for human use in gene therapy. Actually 12 different AAV serotypes (AAV1 to 12) are known, each with different tissue tropisms (Wu, Z Mol Ther 2006; 14:316-27). Recombinant AAV are derived from the dependent parvovirus AAV2 (Choi, V W J Virol 2005; 79:6801-07). The adeno-associated virus type 1 to 12 can be engineered to be replication deficient and is capable of infecting a wide range of cell types and species (Wu, Z Mol Ther 2006; 14:316-27). It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a preferred embodiment, the antisense oligonucleotide, siRNA, shRNA or ribozyme nucleic acid sequence is under the control of a heterologous regulatory region, e.g., a heterologous promoter. The promoter may be specific for the lung cells.

Pharmaceutical Compositions

The inhibitor of Arp 2/3 complex activity or expression may be combined with pharmaceutically acceptable excipients, and optionally sustained-release matrices, such as biodegradable polymers, to form therapeutic compositions.

In the pharmaceutical compositions of the present invention for oral, sublingual, subcutaneous, intramuscular, intravenous, transdermal, local, inhaled or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The Inhibitor of Arp 2/3 complex activity or expression of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The inhibitor of Arp 2/3 complex activity or expression of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

Method of Sreening

The present invention also relates to a method for screening a plurality of candidate compounds useful for treating idiopathic pulmonary fibrosis comprising the steps consisting of (a) testing each of the candidate compounds for its ability to inhibit Arp 2/3 complex activity or expression and (b) and positively selecting the candidate compounds capable of inhibiting said Arp 2/3 complex activity or expression.

Typically, the candidate compound is selected from the group consisting of small organic molecules, peptides, polypeptides or oligonucleotides. Other potential candidate compounds include antisense molecules, siRNAs, or ribozymes.

Testing whether a candidate compound can inhibit Arp 2/3 complex activity or expression can be determined using or routinely modifying reporter assays known in the art.

For example, the method may involve contacting cells expressing Arp 2/3 complex with the candidate compound, and measuring the Arp 2/3 complex mediated transcription (e.g., activation of promoters containing Arp 2/3 complex binding sites), and comparing the cellular response to a standard cellular response. Typically, the standard cellular response is measured in absence of the candidate compound. A decrease cellular response over the standard indicates that the candidate compound is an inhibitor of Arp 2/3 complex activity.

In another embodiment the invention provides a method for identifying a ligand which binds specifically to Arp 2/3 complex. For example, a cellular compartment, such as a membrane or a preparation thereof, may be prepared from a cell that expresses a molecule that binds Arp 2/3 complex. The preparation is incubated with labelled Arp 2/3 complex and complexes of ligand bound to Arp 2/3 complex are isolated and characterized according to routine methods known in the art. Alternatively, the Arp 2/3 complex interacting polypeptide may be bound to a solid support so that binding molecules solubilized from cells are bound to the column and then eluted and characterized according to routine methods. In another embodiment, a cellular compartment, such as a membrane or a preparation thereof, may be prepared from a cell that expresses a molecule that binds Arp 2/3 complex such as a molecule of a signalling or regulatory pathway modulated by Arp 2/3 complex. The preparation is incubated with labelled Arp 2/3 complex in the absence or the presence of a candidate compound. The ability of the candidate compound to bind the binding molecule is reflected in decreased binding of the labelled ligand. Molecules which bind gratuitously, i.e., without inducing the effects of Arp 2/3 complex on binding the Arp 2/3 complex binding molecule, are most likely to be good inhibitor of Arp 2/3 complex activity.

Another method involves screening for compounds which inhibit Arp 2/3 complex activity by determining, for example, the amount of transcription from promoters containing Arp 2/3 complex binding sites in a cell that expresses Arp 2/3 complex. Such a method may involve transfecting a eukaryotic cell with DNA encoding Arp 2/3 complex such that the cell expresses Arp 2/3 complex, contacting the cell with a candidate compound, and determining the amount of transcription from promoters containing Arp 2/3 complex binding sites. A reporter gene (.e.g, GFP) linked to a promoter containing an Arp 2/3 complex binding site may be used in such a method, in which case, the amount of transcription from the reporter gene may be measured by assaying the level of reporter gene product, or the level of activity of the reporter gene product in the case where the reporter gene is an enzyme. A decrease in the amount of transcription from promoters containing Arp 2/3 complex binding sites in a cell expressing Arp 2/3 complex, compared to a cell that is not expressing Arp 2/3 complex, would indicate that the candidate compound is an inhibitor of Arp 2/3 complex activity.

The candidate compounds that have been positively selected may be subjected to further selection steps in view of further assaying its properties on lung fibroblasts isolated from subjects suffering from idiopathic pulmonary. For example, the candidate compounds that have been positively selected with the screening method as above described may be further selected for their ability to inhibit the migration of lung fibroblasts from patients with IPF or to inhibit the expression of collagen 1a1 by lung fibroblasts from patients with IPF. Typically, the screening method may further comprise the steps of i) bringing into contact a lung fibroblast from patients with IPF with a positively selected candidate compound ii) determining the migration of said lung fibroblast or the expression of collagen 1a1 by said fibroblast and iii) comparing the migration or expression determined at step ii) with the migration or expression determined when step i) is performed in the absence of the positively selected candidate compound. Step i) as above described may be performed by adding an amount of the candidate compound to be tested to the culture medium of the fibroblast. Usually, a plurality of culture samples are prepared, so as to add increasing amounts of the candidate compound to be tested in distinct culture samples. Generally, at least one culture sample without candidate compound is also prepared as a negative control for further comparison.

Finally, the candidate compounds that have been positively selected may be subjected to further selection steps in view of further assaying its properties on animal models for IPF. Typically, the positively selected candidate compound may be administered to the animal model and the progression of lung fibrosis is determined and compared with the progression of lung fibrosis in an animal model that was not administered with the candidate compound.

The invention will be further illustrated by the following figures and examples. However, these examples and figures should not be interpreted in any way as limiting the scope of the present invention.

### FIGURES

FIG. 1. Expression of the Arp2/3 complex subunits in human lung mesenchymal cells cultured from patients without chronic parenchymal lung disease (controls, open bars, n=20) and patients with IPF (filled bars, n=20). Data are expressed as z-scores. *: p<0.05 between controls and IPF. Mean and SD. N=20.

FIG. 2. ARPC2 protein is expressed in IPF lung mesenchymal cells. Expression of ARPC2 protein and the endogenous control beta-tubulin in human lung mesenchymal cells cultured from lungs without chronic lung disease (N=8) and lungs with IPF (N=9).

FIG. 3. Expression of Arp2/3 complex subunits at the mRNA level in the lungs of bleomycin- and CK666-treated mice. Data are means +/− SEM. *: p<0.05 versus Saline. N=10-12 per group.

FIG. 4. CK666 inhibited collagen deposition in the lungs of bleomycin-instilled mice. A: Insoluble collagen. B: Procollagen-1 protein. C: Collal mRNA reported to Gapdh mRNA. D: Collagenase activity. E: Lysyl oxidase activity. AU: Arbitrary Units. *: p<0.05 versus Saline. $: p<0.05 versus Bleo. N=10-14 per group.

FIG. 5. Weight loss and BALF proteins. A: Weight variation as a percentage of original weight. B: BALF cells. *: p<0.05 versus Saline. N=10-14 per group.

FIG. 6. Effect of CK666 (100 μM) on lung fibroblast cell growth and G/F actin ratio. A: Fold-Increase in cell number relative to seeding (Day 4). B: G- and F-actin levels in lung fibroblasts treated with CK666 and TGFB1 (5 ng/ml) or controls. Upper panel: Representative Western Blot experiment. Lower panel: G/F actin ratio. *: p<0.05 versus DMSO. $: p<0.05 versus DMSO+TGFB1. N=4.

FIG. 7. Effect of siARPC2 on lung fibroblast cell growth and G/F actin ratio. A: ARPC2 expression in lung fibroblasts transfected with 4 nM of either siARPC2 or siControl. B: Fold-Increase in cell number relative to seeding (Day 4). C: G- and F-actin levels in lung fibroblasts treated with siARPC2 and TGFB1 (5 ng/ml) or controls. Upper panel: Representative Western Blot experiment. Lower panel: G/F actin ratio. *: p<0.05 versus siControl. $: p<0.05 versus siControl+TGFB1. N=4.

FIG. 8. Effect of CK666 on spontaneous and TGFB1-induced ACTA2 expression. A: ACTA2 mRNA (RTPCR). B: ACTA2 protein (cell-based ELISA). *: p<0.05 versus DMSO. $: p<0.05 versus DMSO+TGF. N=5.

FIG. 9. Effect of siARPC2 on spontaneous and TGFB1-induced ACTA2 expression. A: ACTA2 mRNA (RTPCR). B: ACTA2 protein (cell-based ELISA). *: p<0.05 versus siControl. £: p<0.05 versus siARPC2. $: p<0.05 versus siControl+TGF. N=5.

FIG. 10. Effect of CK666 on spontaneous and TGFB1-induced COL1 expression. A: COL1A1 mRNA (RTPCR). B: COL1A2 mRNA (RTPCR). C: COL1 protein (ELISA). Results are relative to DMSO. *: p<0.05 versus DMSO. £: p<0.05 versus CK666. N=5.

FIG. 11: Effect of siARPC2 on spontaneous and TGFB1-induced COL1 expression. A: COL1A1 mRNA (RTPCR). B: COL1A2 mRNA (RTPCR). C: COL1 protein (ELISA). Results are relative to DMSO. *: p<0.05 versus siControl. N=5.

### EXAMPLE

Material & Methods:

Expression of the Arp2/3 Complex in IPF Lung Mesenchymal Cells

Data from a meta-analysis of 4 distinct microarray datasets (24) were used to assess the expression levels of the Arp2/3 subunits ARP2, ARP3, p41/ARPC1, p34/ARPC2, p21/ARPC3, p20/ARPC4, and p16/ARPC5 using two-tailed unpaired t-tests. Lung mesenchymal cells were obtained by primary culture in 6 patients without chronic parenchymal lung disease and 7 patients with IPF (Melboucy-Belkhir et al. 2014). ARPC2 protein expression in lysates of lung mesenchymal cells was determined by Western Blotting with the rabbit polyclonal 07-227 antibody (Merck Millipore, Molsheim, France).

Cell Culture Experiments

Primary human normal lung fibroblasts (CCD-16Lu cells, American Type Culture Collection, Manassas, Va., USA) were grown in DMEM-Glutamax medium supplemented with 10% fetal bovine serum and penicillin/streptomycin (Thermofisher, Villebon-sur-Yvette, France), at 37° C. with 5% CO₂. Experiments were performed between passages 7 and 12, when cells reached 70% confluency. Fibroblasts were treated either with 100 μM CK666 (Tocris, Lille, France) or 1/1000 dimethylsulfoxide (DMSO, Sigma,St Quentin Fallavier, France) as a control. In a second set of experiments, fibroblasts were transfected with 4 nM of either short interfering RNAs targeting ARPC2 (siARPC2, Silencer Select, Thermofisher) or control RNAs (siControl), using the Lipofectamine RNAimax reagent (Thermofisher). For cell growth assays, 50 000 cells/well were plated in 12-well plates, grown for 4 days, detached with 5% trypsin and counted using Kova Glasstic counting slides (Thermofisher). For differentiation assays, cells were plated in 12-well plates, grown to 70% confluency, treated for 24 h with siControl or siARPC2 when appropriate, starved of serum for 24 h, and treated with either CK666 or DMSO 30 minutes before treatment with either 5 ng/ml recombinant human TGFB1 (R&D Systems, Lille, France) of control solvent for 48h. Cell proteins were recovered in 100 μl lysis buffer (50 mM Tris HCl pH7.4, 150 mM NaCl, 0.1% SDS, 0.5% sodium deoxycholate, 1% Triton X-100) with 1% protease inhibitor (P8340, Sigma) and quantitated by the BiCinchoninic acid Assay (Pierce, Thermofisher); mRNA was extracted using the GeneJET RNA Purification Kit (Thermofisher).

Determination of G- and F-Actin Levels

500 000 cells were seeded in 75 cm2 flasks and treated with either CK666 or DMSO for 24 h. Cells were detached with trypsin and incubated for 5 min in 0.1% Triton-X100 in phosphate buffered saline (PBS) with protease inhibitor at room temperature, then centrifuged at 15 000 G for 5 min at 4° C. The pellet was solubilized in lysis buffer. Supernatant (G-actin) and pellet (F-actin) samples were analyzed by immunoblotting on polyvinylidene fluoride membranes using a beta-actin primary antibody (Sigma).

Determination of ACTA2 Protein Levels by Cell-Based ELISA and Immunofluorescence

For cell-based ELISA, cells were grown in 12-well plates, fixed with 4% paraformaldehyde for 15 min, permeabilized with 0.5% Triton X-100 in PBS, blocked with 1% bovine albumin, incubated overnight at 4° C. with an ACTA2 primary antibody (A2547, dilution 1/1000, Sigma), and sequentially incubated with an anti-mouse biotinylated antibody (Vector, dilution 1/400), streptavidin-horseradish peroxidase (R&D Systems), and Tetramethylbenzidine substrate (Sigma). The reaction was stopped with H2504 and absorbance readings were taken at 450 nm with 570 nm as a reference wavelength.

For immunofluorescence, cells were seeded on 8-well Labtek slides, permeabilized, stained with the same antibody, incubated with 1/100 anti-mouse IgGx CruzFluor594 (Santa Cruz Biotechnologies, Santa Cruz, Calif., USA), and counterstained with both 1/1000 Alexa488 phalloidin and 1/1600 Hoescht 33342 reagent (Molecular Probes, Thermofisher). Images were acquired on an Evos FL Cell Imaging System (Thermofisher) at ×20 magnification.

ELISA

Procollagen-1 (COL1) and Glyćeraldéhyde-3-phosphate déshydrogenase (GAPDH) were quantified by ELISA (DY6220 and DYC5718, R&D Systems). COL1 was expressed relative to GAPDH as an endogenous control.

RTPCR Experiments

RNAs were reverse-transcribed using the First Strand cDNA Synthesis Kit (Thermofisher). Transcripts were quantified by RTPCR (Sybr select Master Mix, Thermofisher) and expressed relative to UBC (human samples) or Gapdh (mouse samples) as an endogenous control using the 2^{-ΔCt} method. Primers are listed in Table 1.

Bleomycin-Induced Fibrosis in Mice

Male, 8-week old C57BL6-J mice (Janvier, Le Genest-St-Ile, France) were treated with an orotracheal instillation of either 120 μg bleomycin (Laboratoires Bellon, France) or saline, under isoflurane anesthesia. In a first set of experiments, mice were treated from the 7th to the 14th day following instillation with daily subcutaneous injections of either 5 mg/kg of the Arp2/3 inhibitor CK666 (Tocris Biochemicals, Bristol, UK) in 100 μl of 40% Dimethylsulfoxide in saline (vehicle) (M. Park et al. 2013), or vehicle alone. Mice were monitored and weighted daily and their diet was supplemented with gel-based food (Dietgel Recovery gels, ClearH2O, Westbrook, Me., USA). On the 14th day following bleomycin, mice were anesthetized with 300 mg/kg Ketamine and 30 mg/kg xylazine, and euthanized by exsanguination. Bronchoalveolar lavage was performed with 2×1 ml saline. Lung were either snap-frozen in liquid nitrogen or inflation-fixed in 4% formaldehyde. Inhibition of the Arp2/3 complex in vivo. The study protocol was approved by the regional ethics board (APAFIS#5669-20 16061413402331).

Total insoluble collagen was quantified in left lung homogenates (Sircol Insoluble collagen Assay, Biocolor, Carrickfergus, UK). Soluble procollagen-1 was quantified in homogenates of the right upper lung lobe by ELISA (DY6220, R&D Systems). RNAs were extracted from the right middle lung lobe (Allprep, Qiagen) and reverse-transcribed (First Strand cDNA Synthesis Kit, Thermofisher). Transcripts associated with fibrogenesis were quantified by RTPCR (Sybr select Master Mix, Thermofisher) and expressed relative to Gapdh as an endogenous control. Primers are listed in Table 1. The activity of enzymes key to collagen polymerization and degradation was measured in lysates of right lower lobe using fluorimetric assays specific for lysil oxidase (Amplite™ Fluorimetric Lysyl Oxidase Assay, AAT Bioquest, Sunnyvale, Calif., USA) and collagenase (fluorimetric DQ collagen type 1, Sigma Aldrich, St Quentin-Fallavier, France). Clostridium histolyticum collagenase (Sigma-Aldrich) was used as a positive control for collagenase assays. Total BALF proteins were measured with the BCA protein assay (Thermofisher). BALF cells were counted using a hemocytometer. For histological analysis, 5-μm sections of paraffin-embedded lung were stained with Masson's trichrome stain.

Statistical Analysis

The number of mice per group was calculated based on the effect size observed in previous studies (Fabre et al. 2008). Data are shown as means and standard deviation of the mean. Comparison between groups were performed by Student's t-test with Prism 5 for Windows (Graphpad, San Diego, Calif., USA).

TABLE 2
PCR primers for RTPCR in mice.

| Transcript | Gene name | Forward sequence | Reverse sequence |
|---|---|---|---|
| Gapdh | glyceraldehyde-3- | TGCCCAGAACATCATCCCT | TCAGATCCACGACGGAC |
| | phosphate | G (SEQ ID No1) | ACA (SEQ ID No2) |
| | dehydrogenase | | |
| Arp2 | Actin-related | CACATCTTCCCAGCTTTGG | CAGCTCACTTGCCTCAT |
| | protein 2 | T (SEQ ID No3) | CAC (SEQ ID No4) |
| Arp3 | Actin-related | CAGGCTGTTCTTGCCTTAG | ATCCTTCAGCCACAGGA |
| | protein 3 | C (SEQ ID No5) | ATG (SEQ ID No6) |
| Arpc1α | Actin-related | GAGGATTAACCGTGCAGC | CTTGCTCACCCACCAGT |
| | protein 2/3 | CA (SEQ ID No7) | CAT (SEQ ID No8) |
| | complex, subunit | | |
| | 1 | | |
| Arpc2 | Actin-related | CACCGCCCTTGTTTCTCCT | GCTTCTGGTTTGTTTCCG |
| | protein 2/3 | T (SEQ ID No9) | GC (SEQ ID No10) |
| | complex, subunit | | |
| | 2 | | |
| Arpc3 | Actin-related | CCATTTATGCCAAGCCTGC | GTCCACCACTTGCTGGG |
| | protein 2/3 | G (SEQ ID No11) | TTT (SEQ ID No12) |
| | complex, subunit | | |
| | 3 | | |
| Arpc4 | Actin-related | CTCTCCGCCCCTACCTTAG | TGCTACTCCTGACTTCG |
| | protein 2/3 | T (SEQ ID No13) | ACC (SEQ ID No14) |
| | complex, subunit | | |
| | 4 | | |
| Arpc5 | Actin-related | GAAGGTGGACGTGGACGA | GCTGTCATGTTTCCTTGC |
| | protein 2/3 | AT (SEQ ID No15) | CG (SEQ ID No16) |
| | complex, subunit | | |
| | 5 | | |
| Col1α1 | Collagen 1 alpha | CCAGTGGCGGTTATGACTT | GCTGCGGATGTTCTCAA |
| | 1 | CA (SEQ ID No17) | TCTG (SEQ ID No18) |
| Lox | Lysyl oxidase | AGGGCGGATGTCAGAGAC | AATCCCTGTGTGTGTGC |
| | | TA (SEQ ID No19) | AGT (SEQ ID No20) |
| Lox12 | Lysyl oxidase | CCTCCCTCCCGCTTTCA | CAAGTGTGCAGTCCTGG |
| | like-2 | (SEQ ID No21) | GTTT ((SEQ ID No22) |
| Tg2 | Transglutaminase- | GGAGGAGCGACGGGAATA | ATTCCATCCTCGAACTG |
| | 2 | TG (SEQ ID No23) | CCC (SEQ ID No24) |
| Col1α2 | Collagen 1 alpha | CTGATGGCAGAGCTGGTGT | ATGTTGCCAGCTTCACC |
| | 2 | A (SEQ ID No25) | TCT (SEQ ID No26) |
| UBC | Ubiquitin C | CAGTTGGTCCTGCGCTTG | TTTTTTGGGAATGCAAC |
| | | (SEQ ID No27) | AACTTT (SEQ ID No28) |
| ARPC2 | Actin related | TTTCATCCCGCTAATCTTG | GGGAGAAAAGGGAAGT |
| | protein 2/3 | G (SEQ ID No29) | GAGG (SEQ ID No30) |
| | complex, subunit | | |
| | 2 | | |
| ACTA2 | Actin, Alpha 2, | GAAGAGCATCCCACCCTG | ATTTTCTCCCGGTTGGCC |
| | Smooth Muscle | (SEQ ID No31) | (SEQ ID No32) |
| COL1A1 | Collagen 1 alpha | TTGAGACTCAGCCACCCAG | CAGTTCTTGGCTGGGTG |
| | 1 | AGT (SEQ ID No33) | TTTT (SEQ ID No34) |
| COL1A2 | Collagen 1 alpha | AGCAGGTCCTTGGAAACCT | GAAAAGGAGTTGGACTT |
| | 2 | T (SEQ ID No35) | GGC (SEQ ID No36) |

Results:

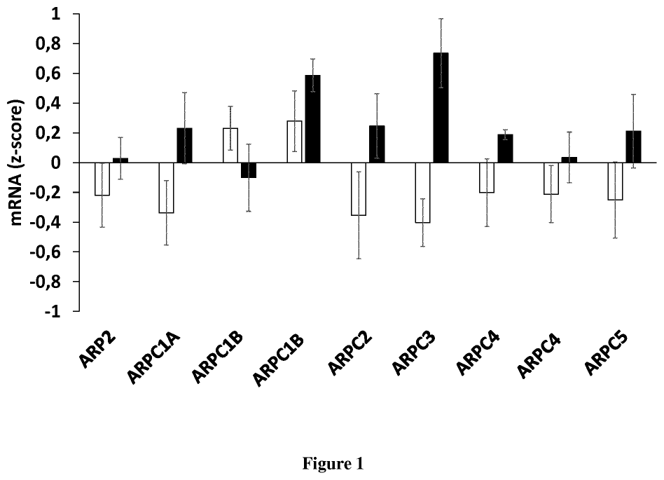

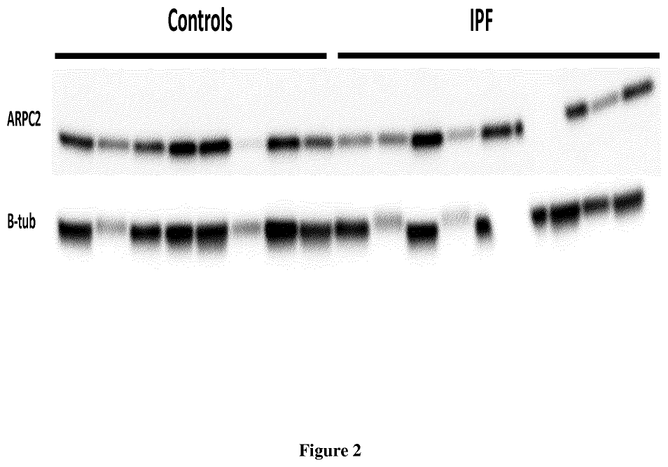

Expression of the Arp2/3 complex in human IPF lung mesenchymal cells Expression values of the protein subunits of the Arp2/3 complex in control and IPF mesenchymal cells were obtained from a meta-analysis of 4 microarray studies comparing the transcriptome of these cells after 4-5 culture passages (Plantier et al. 2016). As shown in FIG. 1, the expression of ARPC2, ARPC3 and ARPC5L was increased in IPF cells, suggesting upregulation of Arp2/3 function. At the protein level, ARPC2 was detected in human lung mesenchymal cells cultured from both lungs with IPF and lungs without chronic parenchymal disease (FIG. 2).

Expression of the Arp2/3 Complex in the Lungs of Bleomycin-Treated Animals

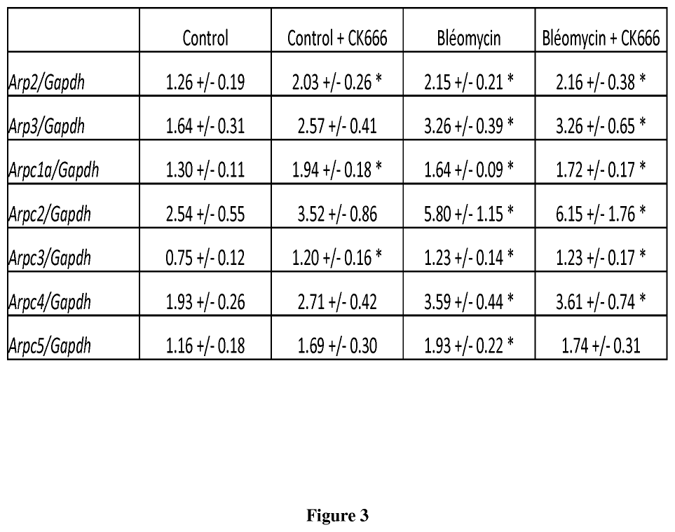

The expression of all Arp2/3 subunits were overexpressed in the lungs of mice following bleomycin instillation at the mRNA level (FIG. 3). The Arp2/3 complex inhibitor CK666 induced increased expression of Arp2, ArpCla and Arpc3, suggesting that the Arp2/3 complex regulates its own expression levels in a negative feedback loop.

Inhibition of the Arp2/3 Complex Inhibited Lung Fibroblast Growth and Increased G/F actin Ratio In Vitro

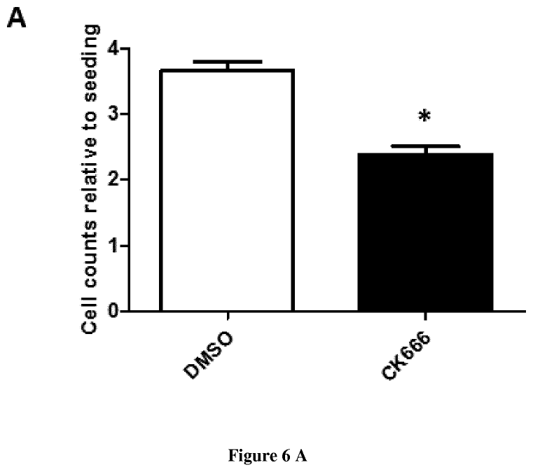

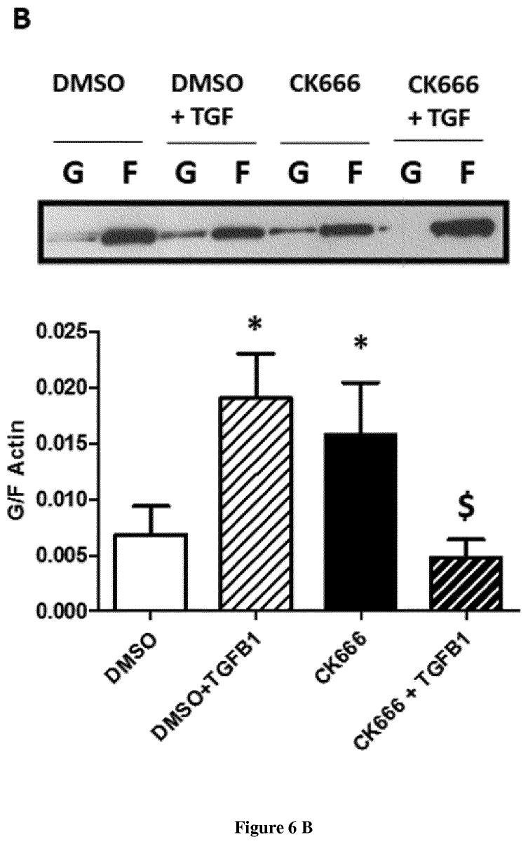

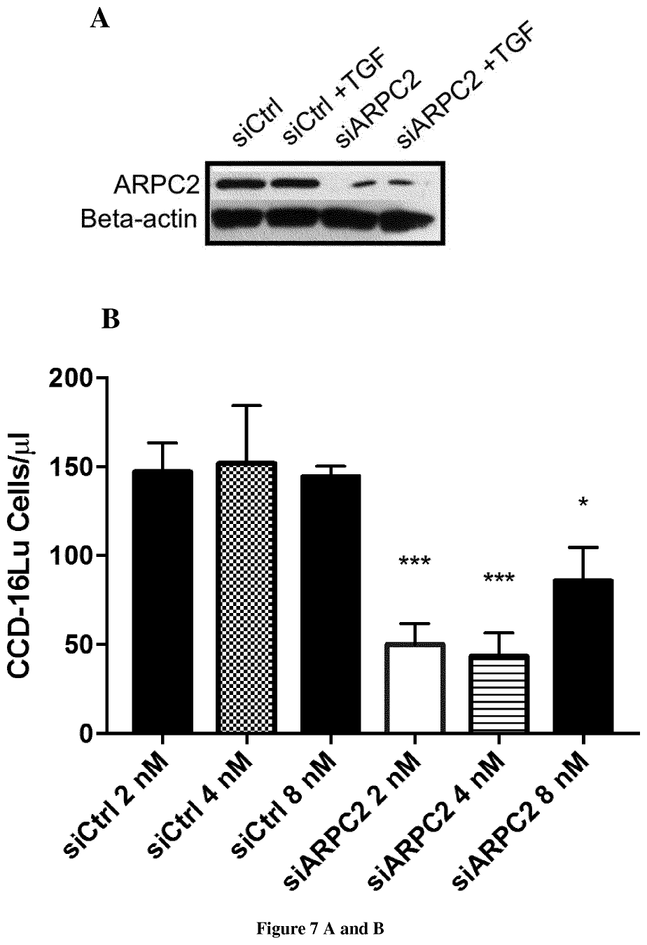

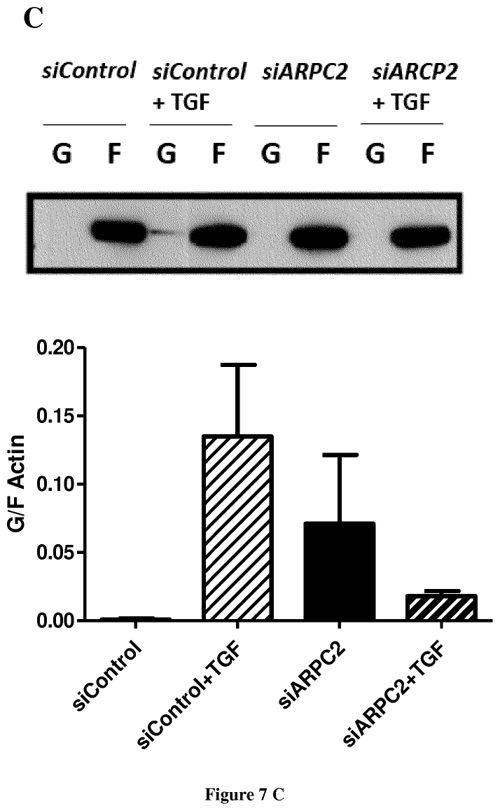

The Arp2/3 complex regulates polymerization of actin and cell proliferation. To verify that CK666 exerted effects consistent with Arp2/3 inhibition in lung fibroblasts, cell growth and the G-actin/F-actin ratio were assessed under CK666 and siARPC2 treatment. CK666 reduced cell growth by 36% (FIG. A6), and increased the G/F actin ratio suggestive of reduced actin polymerization (FIG. 6B). siARPC2 reduced ARPC2 levels by >80% (FIG. 7A) and exerted effects similar to CK666 with regard to cell growth (FIG. 7B) and the G/F actin ratio (FIG. 7C).

Inhibition of Arp2/3 Repressed TGFB1-Induced Actin Remodeling and Myofibroblast Differentiation

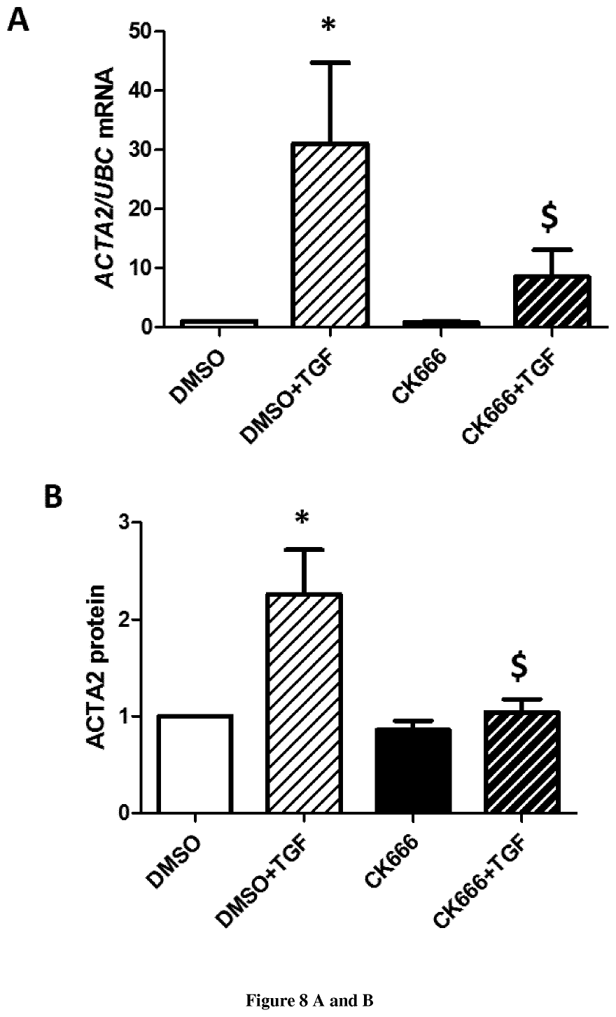

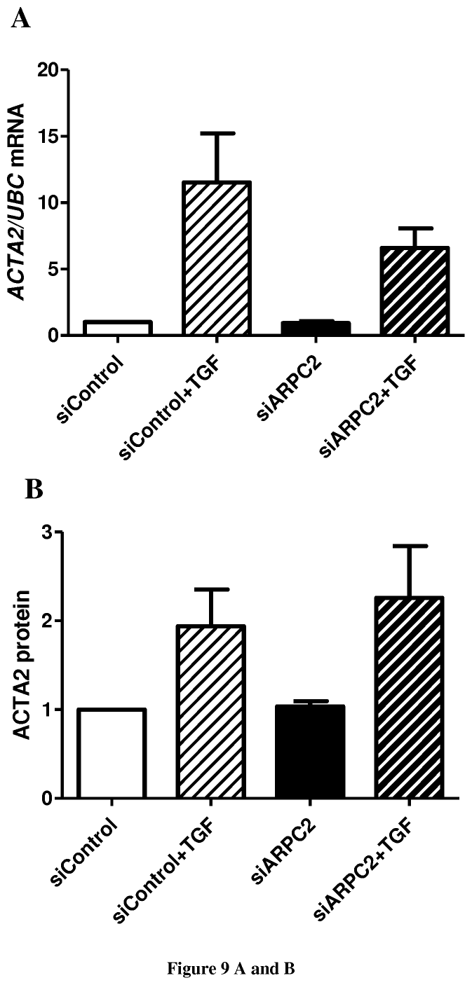

TGFB1 induced an increase in the G/F actin ratio consistent with actin depolymerization. This increase was inhibited by CK666 (FIG. 6B) and siARPC2 (FIG. 7C). CK666 did not modulate expression of ACTA2 mRNA or protein in unstimulated lung fibroblasts (FIG. 8A, 8B, 8C). TGFB1 increased ACTA2 expression at both the mRNA (FIG. 8A) and protein levels (FIG. 8B), and induced formation of ACTA2-positive stress fibers (FIG. 8C). The TGFB1-driven increase in ACTA2 expression was partially (mRNA) or totally (protein) inhibited by CK666. Likewise, siARPC2 reduced the TGFB1-induced increase in ACTA2 mRNA (FIG. 9A), although ACTA2 protein was unchanged (FIG. 9B).

CK666 Reduced Basal Expression of COL1 Protein in Lung Fibroblasts

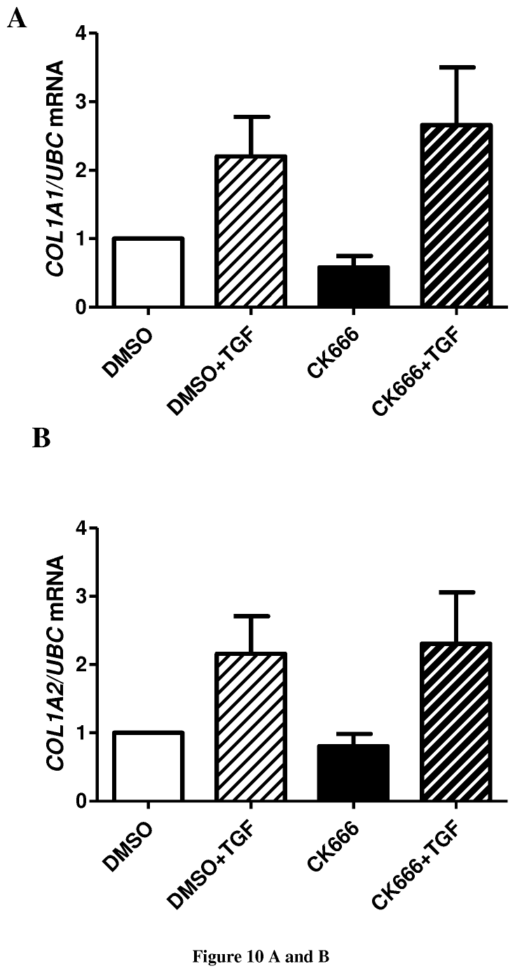

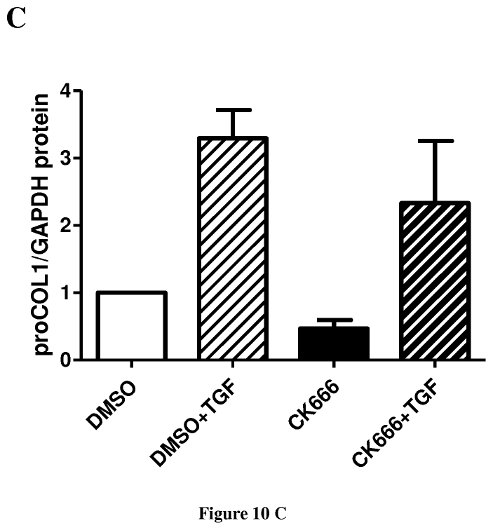

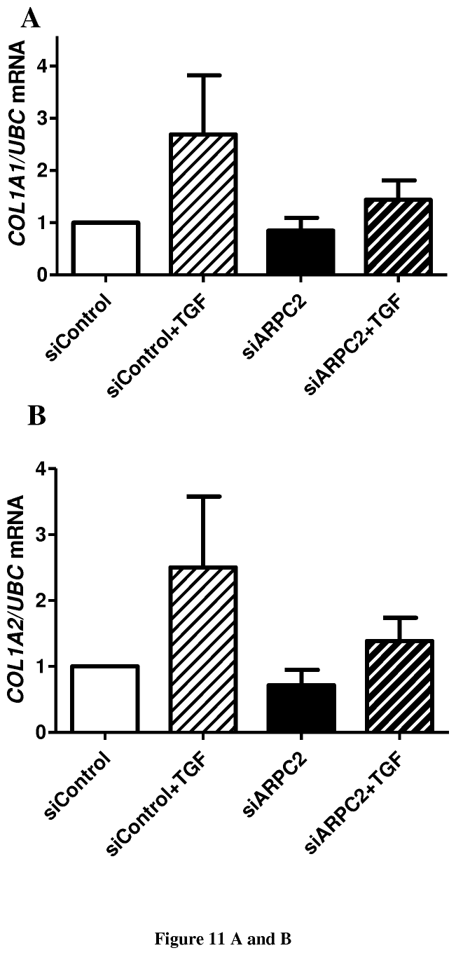

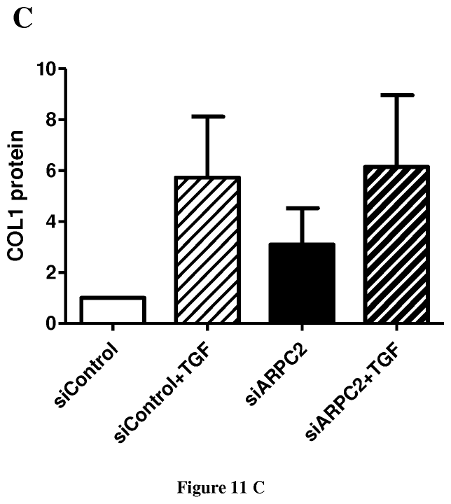

In unstimulated cells, CK666 did not modulate expression of COL1A1 or COL1A2 mRNAs (FIGS. 10A and 10B) but reduced the COL1/GAPDH protein ratio by 54% (FIG. 10C). CK666 did not modulate the TGFB1-driven increase in COL1A1 and COL1A2 mRNAs, although there was a trend towards reduction of the TGFB1-driven increase in COL1 protein (30% reduction, p=0.13). siARCP2 reduced the TGFB1-stimulated expression of COL1A1 mRNA by 40% (FIG. 11A). Neither COL1A2 mRNA nor COL1 protein were reduced by siARPC2 (FIG. 11B and FIG. 11C).

Inhibition of the Arp2/3 Complex with CK666 Prevents the Development of Bleomycin-Induced Lung Fibrosis in Mice

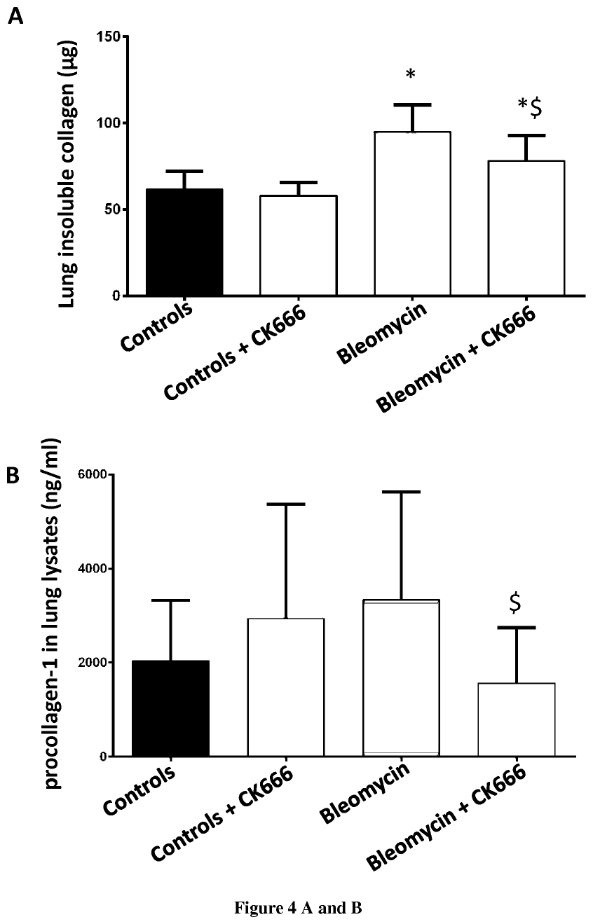

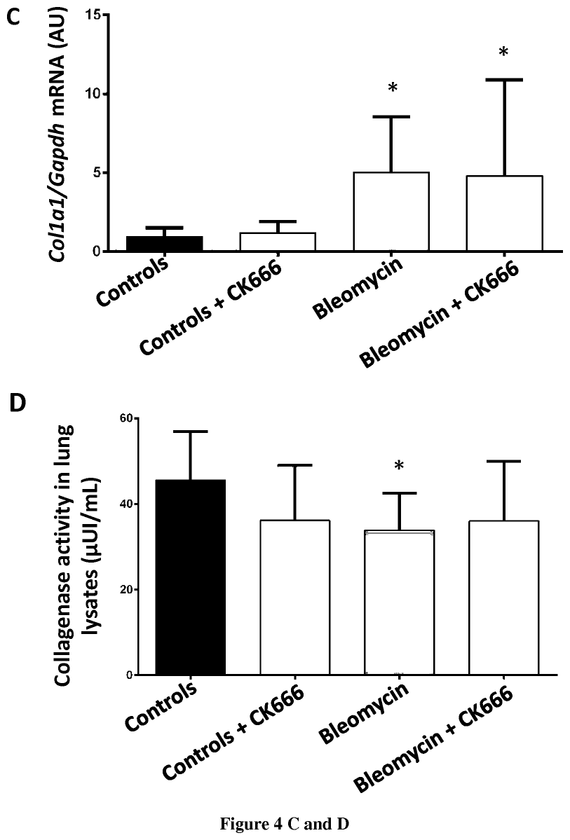

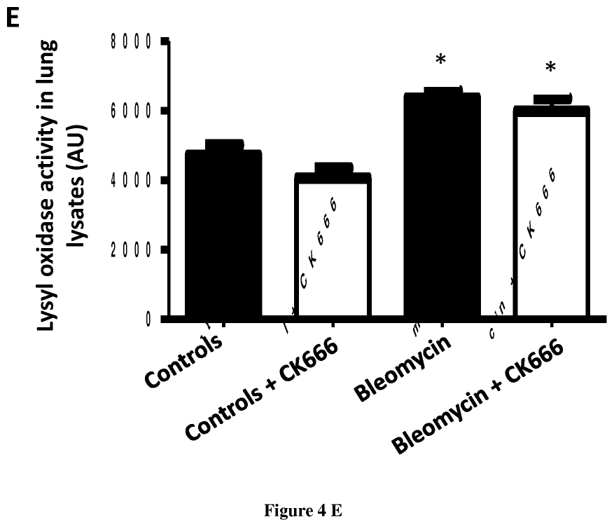

The lungs of mice 14 days after intratracheal instillation of saline and daily IP injections of Vehicle from Day 7 onwards appeared normal by histological analysis. IP CK666 did not result in modifications of lung histology. In mice treated with bleomycin IT and Vehicle, focal lesions were observed (FIG. 4C). These lesions comprised interstitial edema, type 2 pneumocyte hyperplasia, interstitial lymphocyte infiltrates, accumulation of foamy macrophages in alveolar spaces, and destructive fibrosis as shown by distortion of the lung architecture and collagen deposition. In mice treated with CK666 following bleomycin, similar although less severe focal lesions were observed.

Since histological analysis does not allow to precisely quantitate lung fibrosis, the total content of left lungs in insoluble collagen was determined. As shown in FIG. 4A, lung insoluble collagen was reduced by 20% in bleomycin-instilled mice treated with CK666 in comparison with Vehicle-treated animals, indicative of an antifibrotic effect of CK666. The lung content in procollagen-1 was reduced by 55% in CK666 treated mice in comparison with vehicle-treated mice following bleomycin instillation (FIG. 4B). By contrast, CK666 did not reduce the bleomycin-induced increase in lung Collal mRNA (FIG. 4C). For a better understanding of the mechanisms by which CK666 reduced lung collagen deposition, the activity of collagenase and lysyl oxidase in the lung were determined in lung lysates. As shown in FIG. 4D, there was a slight reduction in collagenase activity in the lungs of bleomycin-instilled mice in comparison with saline-instilled mice, and no effect of CK666 in this regard. The bleomycin-induced 2-fold increase in lung lysyl oxidase activity was not modified by CK666 (FIG. 4E). CK666 did not reduced levels of LOX, LOXL2 or TG2 mRNAs in the lungs of bleomycin-instilled mice not shown).

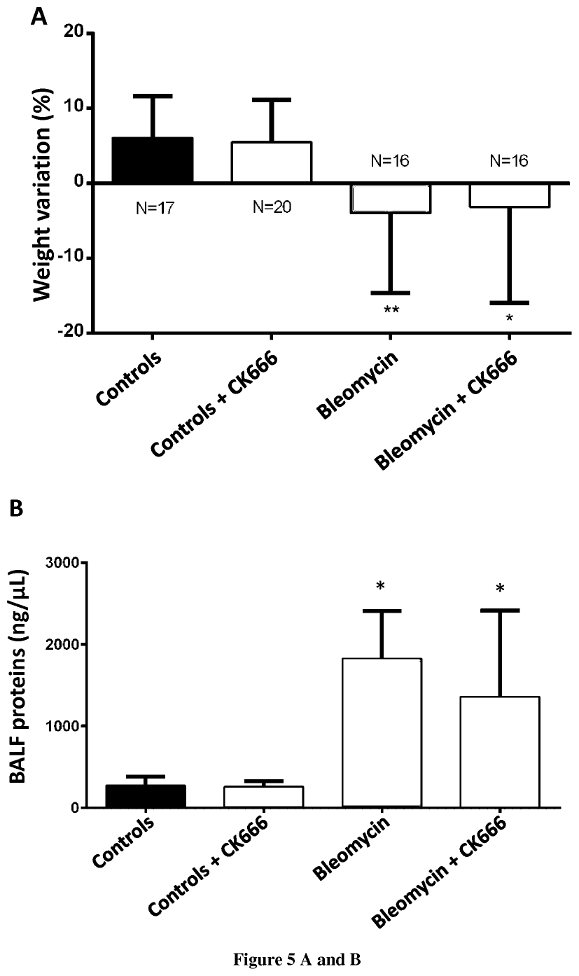

CK666 did not reduce bleomycin-induced weight loss or lung damage Since the bleomycin-induced fibrosis model is associated with significant weight loss and inflammation, and because anti-inflammatory compounds repress fibrosis in this model, it is important to assess whether the protective effect of CK666 against lung fibrosis may be related to properties of the compound unrelated to collagen metabolism. As shown in FIG. 5, CK666 reduced neither weight loss, nor the increase in BALF total protein induced by bleomycin.

Conclusion

This work demonstrates for the first time that the Arp2/3 complex is overexpressed in IPF lung mesenchymal cells, and that the activity of the Arp2/3 complex is required for the accumulation of insoluble collagen in the lungs following bleomycin instillation in mice. The mechanisms by which CK666 prevented collagen accumulation in mouse lung appear directly linked to effects on collagen synthesis. Accumulation of fibrillary collagen depends on 1) synthesis of procollagen-1 from collagen-1 mRNA, 2) polymerization of procollagen catalyzed by lysyl oxidases and transglutaminase 2, and 3) degradation of collagen fibers by collagenases. CK666 treatment was associated with reduced lung levels of procollagen-1 but preserved levels of collagen-1 mRNA. CK666 did not modulate collagen polymerization (enzymatic activity and gene expression) or degradation of collagen fibrils (collagenase activity). In addition, CK666 did not modify markers of systemic (weight loss) or pulmonary (BALF protein levels) inflammation. Taken together, these data indicate that CK666 prevented the development of lung fibrosis either by inhibiting translation or by increasing degradation of collagen-1 mRNA in the lungs.

Altogether, these results indicate the Arp2/3 complex is a key player in lung fibrogenesis in mice and humans, and that inhibition of the Arp2/3 complex is a potent novel strategy for treating lung fibrosis.

Throughout this application, various references describe the state of the art to which this invention pertains. The disclosures of these references are hereby incorporated by reference into the present disclosure.

### REFERENCES

Adegunsoye, Ayodeji, and Mary E. Strek. 2016. “Therapeutic Approach to Adult Fibrotic Lung Diseases.” Chest 150 (6): 1371-86. doi:10.1016/j.chest.2016.07.027.
Barry-Hamilton V, Spangler R, Marshall D, McCauley S, Rodriguez H M, Oyasu M, et al. Allosteric inhibition of lysyl oxidase-like-2 impedes the development of a pathologic microenvironment. Nat Med. 2010;16:1009-17.
Cai G-Q, Chou C-F, Hu M, Zheng A, Reichardt L F, Guan J-L, et al. Neuronal Wiskott-Aldrich syndrome protein (N-WASP) is critical for formation of α-smooth muscle actin filaments during myofibroblast differentiation. Am J Physiol Lung Cell Mol Physiol. 2012;303:L692-702.
Canestaro, William J., Sara H. Forrester, Ganesh Raghu, Lawrence Ho, and Beth E. Devine. 2016. “Drug Treatment of Idiopathic Pulmonary Fibrosis: Systematic Review and Network Meta-Analysis.” Chest 149 (3): 756-66. doi:10.1016/j.chest.2015.11.013.
Cheng T, Liu Q, Zhang R, Zhang Y, Chen J, Yu R, et al. Lysyl oxidase promotes bleomycin-induced lung fibrosis through modulating inflammation. J Mol Cell Biol. 2014;6:506-15.
Crestani, Bruno, Valerie Besnard, Laurent Plantier, Keren Borensztajn, and Arnaud Mailleux. 2013. “Fibroblasts: The Missing Link between Fibrotic Lung Diseases of Different Etiologies?” Respiratory Research 14: 81. doi:10.1186/1465-9921-14-81.
Dang, Irene, Roman Gorelik, Carla Sousa-Blin, Emmanuel Derivery, Christophe Guérin, Joern Linkner, Maria Nemethova, et al. 2013. “Inhibitory Signalling to the Arp2/3 Complex Steers Cell Migration.” Nature 503 (7475): 281-84. doi:10.1038/nature12611.
Dupont, Sirio, Leonardo Morsut, Mariaceleste Aragona, Elena Enzo, Stefano Giulitti, Michelangelo Cordenonsi, Francesca Zanconato, et al. 2011. “Role of YAP/TAZ in Mechanotransduction.” Nature 474 (7350): 179-83. doi:10.1038/nature10137.
Duscher, Dominik, Zeshaan N. Maan, Victor W. Wong, Robert C. Rennert, Michael Januszyk, Melanie Rodrigues, Michael Hu, et al. 2014. “Mechanotransduction and Fibrosis.” Journal of Biomechanics 47 (9): 1997-2005. doi:10.1016/j.jbiomech.2014.03.031.
“ERS White Book.” n.d. http://www.erswhitebook.org/.
Fabre, A., J. Marchal-Somm6é, S. Marchand-Adam, C. Quesnel, R. Borie, M. Dehoux, C. Rufflé, et al. 2008. “Modulation of Bleomycin-Induced Lung Fibrosis by Serotonin Receptor Antagonists in Mice.” The European Respiratory Journal 32 (2): 426-36. doi:10.1183/09031936.00126907.
Hetrick, Byron, Min Suk Han, Luke A. Helgeson, and Brad J. Nolen. 2013. “Small Molecules CK-666 and CK-869 Inhibit Actin-Related Protein 2/3 Complex by Blocking an Activating Conformational Change.” Chemistry & Biology 20 (5): 701-12. doi:10.1016/j.chembio1.2013.03.019.
Huang, Jingang, Christian Beyer, Katrin Palumbo-Zerr, Yun Zhang, Andreas Ramming, Alfiya Distler, Kolja Gelse, et al. 2016. “Nintedanib Inhibits Fibroblast Activation and Ameliorates Fibrosis in Preclinical Models of Systemic Sclerosis.” Annals of the Rheumatic Diseases 75 (5): 883-90. doi:10.1136/annrheumdis-2014-207109.
Huang, Steven K, Aaron S Fisher, Anne M Scruggs, Eric S White, Cory M Hogaboam, Bruce C Richardson, and Marc Peters-Golden. 2010. “Hypermethylation of PTGER2 Confers Prostaglandin E2 Resistance in Fibrotic Fibroblasts from Humans and Mice.” The American Journal of Pathology 177 (5): 2245-55. doi:10.2353/ajpath.2010.100446.
Idiopathic Pulmonary Fibrosis Clinical Research Network, Ganesh Raghu, Kevin J. Anstrom, Talmadge E. King, Joseph A. Lasky, and Fernando J. Martinez. 2012. “Prednisone, Azathioprine, and N-Acetylcysteine for Pulmonary Fibrosis.” The New England Journal of Medicine 366 (21): 1968-77. doi:10.1056/NEJMoa1113354.
Johnson, Laura A, Eva S Rodansky, Andrew J Haak, Scott D Larsen, Richard R Neubig, and Peter D R Higgins. 2014. “Novel Rho/MRTF/SRF Inhibitors Block Matrix-Stiffness and TGF-β-Induced Fibrogenesis in Human Colonic Myofibroblasts.” Inflammatory Bowel Diseases 20 (1): 154-65. doi:10.1097/01.MIB.0000437615.98881.31.
Khalil, N, R N O′Connor, H W Unruh, P W Warren, K C Flanders, A Kemp, O H Bereznay, and A H Greenberg. 1991. “Increased Production and Immunohistochemical Localization of Transforming Growth Factor-Beta in Idiopathic Pulmonary Fibrosis.” American Journal of Respiratory Cell and Molecular Biology 5 (2): 155-62.
Leppäranta, Outi, Carla Sens, Kaisa Salmenkivi, Vuokko L. Kinnula, Jorma Keski-Oja, Marjukka Myllarniemi, and Katri Koli. 2012. “Regulation of TGF-βStorage and Activation in the Human Idiopathic Pulmonary Fibrosis Lung.” Cell and Tissue Research 348 (3): 491-503. doi:10.1007/s00441-012-1385-9.
Marinković, Aleksandar, Fei Liu, and Daniel J Tschumperlin. 2013. “Matrices of Physiologic Stiffness Potently Inactivate Idiopathic Pulmonary Fibrosis Fibroblasts.” American Journal of Respiratory Cell and Molecular Biology 48 (4): 422-30. doi:10.1165/rcmb.2012-03350C.
Melboucy-Belkhir, Sara, Pauline Pradère, Sara Tadbiri, Stéfanie Habib, Antoine Bacrot, Stéphanie Brayer, Bernard Mari, et al. 2014. “Forkhead Box Fl Represses Cell Growth and Inhibits COL1 and ARPC2 Expression in Lung Fibroblasts in Vitro.” American Journal of Physiology. Lung Cellular and Molecular Physiology 307 (11): L838-847. doi:10.1152/ajplung.00012.2014.
Noble, Paul W., Carlo Albera, Williamson Z. Bradford, Ulrich Costabel, Marilyn K. Glassberg, David Kardatzke, Talmadge E. King, et al. 2011. “Pirfenidone in Patients with Idiopathic Pulmonary Fibrosis (CAPACITY): Two Randomised Trials.” Lancet (London, England) 377 (9779): 1760-69. doi:10.1016/S0140-6736(11)60405-4.
Olsen K C, Sapinoro R E, Kottmann R M, Kulkarni A A, Iismaa S E, Johnson G V W, et al. Transglutaminase 2 and its role in pulmonary fibrosis. Am J Respir Crit Care Med. 2011;184:699-707.
Park, Minseon, Hyun-Jung Kim, Brian Lim, Adam Wylegala, and Michal Toborek. 2013. “Methamphetamine-Induced Occludin Endocytosis Is Mediated by the Arp2/3 Complex-Regulated Actin Rearrangement.” The Journal of Biological Chemistry 288 (46): 33324-34. doi:10.1074/jbc.M113.483487.
Park, So-Yon, Jung-Chul Park, Min-Soo Kim, Sung-Eun Lee, Ki-Joon Kim, Byung-Joo Jung, Wonse Park, Dong-Won Jeon, Kyoo-Sung Cho, and Chang-Sung Kim. 2015. “Differential Effect of Water-Soluble Chitin on Collagen Synthesis of Human Bone Marrow Stem Cells and Human Periodontal Ligament Stem Cells.” Tissue Engineering. Part A 21 (3-4): 451-62. doi:10.1089/ten.TEA.2014.0156.
Pellegrin, Stéphanie, and Harry Mellor. 2007. “Actin Stress Fibres.” Journal of Cell Science 120 (Pt 20): 3491-99. doi:10.1242/jcs.018473.
Plantier, Laurent, Hélène Renaud, Renaud Respaud, Sylvain Marchand-Adam, and Bruno Crestani. 2016. “Transcriptome of Cultured Lung Fibroblasts in Idiopathic Pulmonary Fibrosis: Meta-Analysis of Publically Available Microarray Datasets Reveals Repression of Inflammation and Immunity Pathways.” International Journal of Molecular Sciences 17 (12). doi:10.3390/ijms17122091.
Pottier, Nicolas, Christelle Cauffiez, Michael Perrais, Pascal Barbry, and Bernard Mari. 2014. “FibromiRs: Translating Molecular Discoveries into New Anti-Fibrotic Drugs.” Trends in Pharmacological Sciences 35 (3): 119-26. doi:10.1016/j.tips.2014.01.003.
Profilin-1 serves as a gatekeeper for actin assembly by Arp2/3-dependent and -independent pathways.—PubMed—NCBI. https://www.ncbi.nlm.nih.gov/pubmed/?term=Profilin-1+serves+as+a+gatekeeper+for+actin+assembly+by+Arp2%2F3-dependent+and+%E2%80%93independent+pathways. Accessed 29 Nov. 2017.
Richeldi, Luca, Roland M. du Bois, Ganesh Raghu, Arata Azuma, Kevin K. Brown, Ulrich Costabel, Vincent Cottin, et al. 2014. “Efficacy and Safety of Nintedanib in Idiopathic Pulmonary Fibrosis.” The New England Journal of Medicine 370 (22): 2071-82. doi:10.1056/NEJMoa1402584.
Sandilands, Emma, Shiva Akbarzadeh, Anna Vecchione, David G. McEwan, Margaret C. rame, and John K. Heath. 2007. “Src Kinase Modulates the Activation, Transport and Signalling Dynamics of Fibroblast Growth Factor Receptors.” EMBO Reports 8 (12): 1162-69. doi:10.1038/sj.embor.7401097.
Selman, Moisés, and Annie Pardo. 2006. “Role of Epithelial Cells in Idiopathic Pulmonary Fibrosis: From Innocent Targets to Serial Killers.” Proceedings of the American Thoracic Society 3 (4): 364-72. doi:10.1513/pats.200601-003TK.
Shiratsuchi, Eri, Megumi Ura, Misako Nakaba, Iori Maeda, and Kouji Okamoto. 2010. “Elastin Peptides Prepared from Piscine and Mammalian Elastic Tissues Inhibit Collagen-Induced Platelet Aggregation and Stimulate Migration and Proliferation of Human Skin Fibroblasts.” Journal of Peptide Science: An Official Publication of the European Peptide Society 16 (11): 652-58. doi:10.1002/psc.1277.
Simon, Dan N., and Katherine L. Wilson. 2011. “The Nucleoskeleton as a Genome-Associated Dynamic ‘Network of Networks.’” Nature Reviews. Molecular Cell Biology 12 (11): 695-708. doi:10.1038/nrm3207.
Suraneni P, Rubinstein B, Unruh J R, Durnin M, Hanein D, Li R. The Arp2/3 complex is required for lamellipodia extension and directional fibroblast cell migration. J Cell Biol. 2012;197:239-51.
Tang W, Cai P, Huo W, Li H, Tang J, Zhu D, et al. Suppressive action of miRNAs to ARP2/3 complex reduces cell migration and proliferation via RAC isoforms in Hirschsprung disease. J Cell Mol Med. 2016;20:1266-75.
Tang Y, Zhao J, Shen L, Jin Y, Zhang Z, Xu G, et al. ox-LDL induces endothelial dysfunction by promoting Arp2/3 complex expression. Biochem Biophys Res Commun. 2016;475:182-8.
To, Ciric, Brian H. Shilton, and Gianni M. Di Guglielmo. 2010. “Synthetic Triterpenoids Target the Arp2/3 Complex and Inhibit Branched Actin Polymerization.” The Journal of Biological Chemistry 285 (36): 27944-57. doi:10.1074/jbc.M110.103036.
Wu, Congying, Sreeja B. Asokan, Matthew E. Berginski, Elizabeth M. Haynes, Norman E. Sharpless, Jack D. Griffith, Shawn M. Gomez, and James E. Bear. 2012. “Arp2/3 Is Critical for Lamellipodia and Response to Extracellular Matrix Cues but Is Dispensable for Chemotaxis.” Cell 148 (5): 973-87. doi:10.1016/j.cell.2011.12.034.
Zhao, Xiao-Han, Carol Laschinger, Pam Arora, Katalin Szászi, Andras Kapus, and Christopher A McCulloch. 2007. “Force Activates Smooth Muscle Alpha-Actin Promoter Activity through the Rho Signaling Pathway.” Journal of Cell Science 120 (Pt 10): 1801-9. doi:10.1242/jcs.001586.
Zhou, Yong, Xiangwei Huang, Louise Hecker, Deepali Kurundkar, Ashish Kurundkar, Hui Liu, Tong-Huan Jin, Leena Desai, Karen Bernard, and Victor J Thannickal. 2013. “Inhibition of Mechanosensitive Signaling in Myofibroblasts Ameliorates Experimental Pulmonary Fibrosis.” The Journal of Clinical Investigation 123 (3): 1096-1108. doi:10.1172/JCI66700.

1. A method for treating a Fibrotic interstitial lung diseases disease (ILD) in a subject in need thereof, comprising
administering to the subject a therapeutically effective amount of an inhibitor of Arp2/3 complex activity or expression.

2. The method of claim 1, wherein the Fibrotic interstitial lung disease (ILD) is selected from the group consisting of Idiopathic pulmonary fibrosis (IPF), idiopathic non specific interstitial pneumonia (NSIP), fibrotic ILD associated with inflammatory rheumatic disease, fibrotic ILD associated with autoimmune connective tissue disease, Sjögren's syndrome, undifferentiated connective tissue disease, and chronic hypersensitivity pneumonitis.

3. The method of claim 2, wherein the Fibrotic interstitial lung diseases (ILD) is Idiopathic pulmonary fibrosis (IPF).

4. The method of claim 1, wherein said inhibitor of Arp2/3 complex activity is a small organic molecule or a biological molecule.

5. The method of claim 4, wherein the small organic molecule is selected from the group consisting of CK-636 , CK-548 , CK666; CK-869 and a synthetic triterpenoid.

6. The method of claim 4 wherein the biological molecule is Arpin or derived peptide.

7. The method of claim 1, wherein the inhibitor of Arp2/3 complex expression is an inhibitor of expression of at least one subunit selected from the group consisting of Arp2, Arp3, p41/ARPC1, p34/ARPC2, p21/ARPC3, p20/ARPC4 and p16/ARPC5

8. The method of claim 7 wherein the inhibitor of Arp2/3 complex expression is an inhibitor of expression of p34/ARPC2.

9. A method for screening a plurality of candidate compounds for use in treating Fibrotic interstitial lung diseases (ILD) comprising the steps of (a) testing each of the plurality of candidate compounds for its ability to inhibit Arp2/3 complex activity or expression and (b) and positively selecting candidate compounds capable of inhibiting Arp2/3 complex activity or expression.

10. A pharmaceutical composition for use in a method for treating Fibrotic interstitial lung diseases (ILD) in a subject in need thereof, comprising an inhibitor of Arp2/3 complex activity or expression.

11. The method of claim 2, wherein the fibrotic ILD associated with inflammatory rheumatic disease is rheumatoid arthritis.

12. The method of claim 2, wherein the fibrotic ILD associated with autoimmune connective tissue disease is systemic sclerosis.

13. The method of claim 5, wherein the synthetic triterpenoid is 2-cyano-3,12-dioxooleana-1,9-dien-28-oic acid (CDDO)-Im or CDDO-Me.
